(19) United States
(12) Patent Application Publication
(10) Pub. No.: US 2005/0202508 A1
(43) Pub. Date: Sep. 15, 2005

(54) Biomarker for Parkinson's disease

(76) Inventor: Guilio M. Pasinetti, New York, NY (US)

Correspondence Address: COOPER & DUNHAM, LLP, 1185 AVENUE OF THE AMERICAS, NEW YORK, NY, 10036, US

(21) Appl. No.: 10/987,710

(22) Filed: Nov. 12, 2004

Related U.S. Application Data
(60) Provisional application No. 60/519,843, filed on Nov. 12, 2003.

(51) Int. Cl. G01N 33/53

(52) U.S. Cl. 435/7.1; 435/7.5

(57) ABSTRACT

This invention provides methods for assessing the likelihood that a patient is suffering from Parkinson's disease by detecting a biomarker in a sample from the patient. This invention also provides diagnostic kits for the same.

This application claims the benefit of U.S. Provisional Application No. 60/519,843, filed on Nov. 12, 2003, the contents of which are hereby incorporated by reference.

### BACKGROUND OF THE INVENTION

Parkinson's disease (PD), characterized by tremor, bradykinesis, rigidity, and postural instability, is a common, progressive neurodegenerative disease affecting nearly 1.5 million Americans. The cost of PD in the U.S. exceeds $5.6 billion annually. The predominant pathologic hallmark of PD is the loss of dopaminergic neurons in the substantia nigra (SN) pars compacta and Lewy bodies. Multiple factors are implicated in PD pathogenesis including genetic predisposition, increased deposition of heavy metals (i.e. iron and manganese) in the basal ganglia, increased oxidative stress combined with reduction of mitochondrial respiratory chain activity, and excitotoxicity 6-12. While there is presently no cure for the degenerative effects of PD, there are effective treatments that provide relief from PD symptoms, without addressing etiological causes of PD. For example, most drug treatments pharmacologically increase the amount of dopamine. The most commonly prescribed drug, L-dopa, is relatively effective and extends the lifespan of PD patients by one year, on average. However, both pharmacological (and surgical) interventions yield limited and temporary benefits. Given that a great deal of evidence suggests that major neurodegeneration is already rampant in the brain before PD motor symptoms are clinically apparent, a tremendous effort is currently underway to identify predictive biological indices of early PD that clearly and specifically identify PD, even in the absence of overt, definitive clinical symptoms.

### SUMMARY OF THE INVENTION

This invention provides a method of assessing the likelihood that a patient is suffering from Parkinson's disease which comprises:
(a) obtaining a fluid sample from the subject;
(b) contacting the sample with an agent which forms a complex with a protein comprising consecutive amino acids having the sequence set forth in SEQ ID NO:1, 2, or 4, or a fragment thereof, under conditions permitting any such protein or fragment thereof present in the sample to complex with the agent; and
(c) detecting the presence of any protein-agent complex formed in step (b),
   wherein the detection of protein-agent complex in step (c) indicates that the patient is likely suffering from Parkinson's disease.

This invention also provides a method of assessing the likelihood that a patient is susceptible to suffering from Parkinson's disease which comprises:
(a) obtaining a fluid sample from the subject;
(b) contacting the sample with an agent which forms a complex with a protein comprising consecutive amino acids having the sequence set forth in SEQ ID NO: 1, 2, or 4, or a fragment thereof, under conditions permitting any such protein or fragment thereof present in the sample to complex with the agent; and
(c) detecting the presence of any protein-agent complex formed in step (b),
   wherein the detection of protein-agent complex in step (c) indicates that the patient is likely susceptible to suffering from Parkinson's disease.

This invention also provides a method of assessing the likelihood that a patient is suffering from Parkinson's disease which comprises:
(a) obtaining a fluid sample from the subject;
(b) contacting the sample with an agent which forms a complex with a human dermcidin-1 (DCD-1) protein comprising consecutive amino acids having the sequence set forth in SEQ ID NO: 1, 2, or 4 or homolog thereof, under conditions permitting any such protein or homolog thereof present in the sample to complex with the agent; and
(c) detecting the presence of any protein-agent complex formed in step (b),

wherein the detection of protein-agent complex in step (c) indicates that the patient is likely susceptible to suffering from Parkinson's disease.

This invention also provides a method of assessing the likelihood that a patient is suffering from Parkinson's disease which comprises:
(a) providing a solid support to which an agent which forms a complex with a human Dermcidin-1 (DCD-1) protein comprising consecutive amino acids having the sequence set forth in SEQ ID NO: 1, 2, or 4, under conditions permitting any human DCD-1 protein present in the sample to complex with the agent is bound;
(b) contacting the solid support from (a) with a fluid sample from the subject;
(c) removing any of the human DCD-1 protein which is not bound to the solid support; and
(d) detecting the presence of protein bound to the solid support,
   wherein the detection of the human DCD-1 protein bound to the solid support in step (d) indicates that the patient is likely suffering from Parkinson's disease.

This invention provides a method of assessing the likelihood that a patient is suffering from Parkinson's disease which comprises:
(a) obtaining a fluid sample from the subject;
(b) contacting the sample with an agent which forms a complex with a protein encoded by a nucleic acid comprising consecutive nucleotides having the sequence set forth in SEQ ID NO:3, or fragment of the protein, under conditions permitting any such protein or fragment thereof present in the sample to complex with the agent; and
(c) detecting the presence of any protein-agent complex formed in step (b),
   wherein the detection of protein-agent complex in step (c) indicates that the patient is likely suffering from Parkinson's disease.

This invention also provides a diagnostic kit which comprises a container comprising a solid support to which an agent which forms a complex with a human dermcidin protein comprising consecutive amino acids having the sequence set forth in SEQ ID NO: 1, 2, or 4 is bound, which agent is labeled with a detectable marker.

### BRIEF DESCRIPTION OF THE FIGURES

FIG. 1: Sequence identification of the 4.6 kDa Parkinson's Disease (PD) cerebreospinal fluid (CSF) biomarker as a processed form of DCD precursor protein.

FIG. 2: DCD immunoreactive material is selectively elevated in the CSF of PD but not probable Alzheimer's Disease (AD) or Amyotrophic Lateral Sclerosis (ALS) cases.

FIG. 3: MPTP-mediated substantia nigra dopaminergic (DAergic) neurotoxicity in mice is associated with elevation of a 4.6 kDa DCD like protein species in the CSF.

FIG. 4: High-resolution in vivo MRM of whole mouse brain.

FIG. 5: Table of other biomarkers.

FIG. 6: Investigation of a cationic protein species as novel biomarker of PD.

FIG. 7: Elevated expression of cystatin C in the CSF of PD and probable AD.

FIG. 8: In situ staining for DCD receptor in normal human midbrain SN tissue.

FIG. 9: The PD biomarker DCD-1 promotes neurotoxicity in SY5Y cells in a dose dependent manner.

FIG. 10. Nucleic acid sequence (SEQ ID NO:3) encoding a human dermcidin.

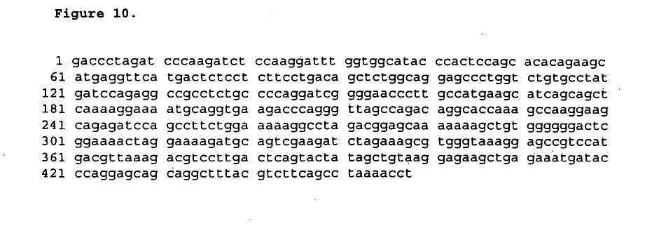

FIG. 11: Selective elevation of Dermicidin/PIF immunoreactivity in the CSF of non-medicated PD cases relative to neurologically normal control cases. % Absorbance at 450 nm was measured with Coulter microplate reader. Data are shown as scatter plot; *P<0.05 vs. neurological controls. Abbreviations: PD, Parkinson's disease; PSP, Progressive Supranuclear Palsy.

FIG. 12: Neuronal PIF mRNA expression is induced in response to MPP+ treatment while exogenous hrPIF (human recombinant full length dermicidin) promotes MPP+ toxicity coincidental with inhibition of MAP kinase signal transduction. Values represent means±SEM of determinations made in 2-3 separate culture preparations; n=3-4 per culture. *P<0.05, ***P<0.01 vs. untreated control group.

FIG. 13: hrPIF (human recombinant full length dermicidin) promotes soluble oligomeric α-synuclein protofibrils in enriched DAergic neuron cultures. Values reflects the sum of α-synuclein immunoreactive tetramer, dimer and monomer forms detected in each sample which are shown as means±SEM of determinations made in 4 separate culture preparations; n=3-5 per culture. *P<0.05, **P<0.001 vs. untreated control group (0 hrPIF).

FIG. 14A-14E: Overexpression human (h)PIF (human full length dermicidin) in transgenic mice promotes α-synuclein expression in the brain. In panel 14A, schematic representation of hPIF transgenic construct comprised of a cytomegalovirus enhancer, followed sequentially by the chicken β-actin promoter, chicken β-actin intron, the hPIF cDNA and a bovine globin poly-adenylation signal sequence. Panel 14B, identification of a transgenic hPIF (TgPIF20) founder mouse by tail DNA dot-blot hybridization. Panel 14C, western blot analysis using rabbit-anti human PIF53-64 antibody from our lab (1:5000) confirmed increased steady state levels of hPIF protein content in serum of 5 month old TgPIF transgenic compared to age matched WT littermates. In Panel 14D, total α-synuclein (tetramer) immunoreactivity in the midbrain and cerebral cortex of TgPIF and WT littermates were quantified densitometrically in Panel 14E.

FIG. 15A-15C: In situ PIF (dermicidin) receptor binding activity in human brain. In 15A, 10 μm semi-adjacent frozen tissue sections encompassing the midbrain substantia nigra, lateral hypothalamus and locus ceruleus from neurological controls¹⁵ were incubated with AP-PIF fusion protein or control AP-reporter (or stained with hematoxilin & eosin (H&E) . In the top panels, faint background staining following control AP-reporter vector incubation; middle panels, AP-PIF staining indicative PIF receptor binding activity; lower panels, semi-adjacent tissue section to each respective brain regions were H&E stained. In 15B, AP-PIF receptor binding or control AP signal in the neuronal SH-SY5Y cell line was further characterized by Schatchard analysis as previously described in other cell types¹⁵. Abbreviations: AP, alkaline phosphatase—reporter; AP-PIF, (AP)—proteolysis inducing factor fusion protein. In 15C the binding concentration of 30,000 PIF receptors per cell is shown.

### DETAILED DESCRIPTION OF THE INVENTION

Definitions

As used herein, and unless stated otherwise, each of the following terms shall have the definition set forth below.

The following abbreviations shall have the meanings set forth below: “A” shall mean Adenine; “bp” shall mean base pairs; “C” shall mean Cytosine; “DNA” shall mean deoxyribonucleic acid; “G” shall mean Guanine.

As used herein, “Dermcidin” is alternatively known as proteolysis inducing factor, or PIF, DCD, and hDCD when referring to human DCD. Dermicidin forms include dermicidn precursor protein (DCD) and dermicidin processed protein (DCD)-1, and fragments of each thereof.

Embodiments of the Invention

This invention provides a method of assessing the likelihood that a patient is suffering from Parkinson's disease which comprises:
(a) obtaining a fluid sample from the subject;
(b) contacting the sample with an agent which forms a complex with a protein comprising consecutive amino acids having the sequence set forth in SEQ ID NO: 1, 2, or 4, or a fragment thereof, under conditions permitting any such protein or fragment thereof present in the sample to complex with the agent; and
(c) detecting the presence of any protein-agent complex formed in step (b),
   wherein the detection of protein-agent complex in step (c) indicates that the patient is likely suffering from Parkinson's disease.

This invention also provides a method of assessing the likelihood that a patient is susceptible to suffering from Parkinson's disease which comprises:
(a) obtaining a fluid sample from the subject;
(b) contacting the sample with an agent which forms a complex with a protein comprising consecutive amino acids having the sequence set forth in SEQ ID NO: 1, 2, or 4, or a fragment thereof, under conditions permitting any such protein or fragment thereof present in the sample to complex with the agent; and
(c) detecting the presence of any protein-agent complex formed in step (b),
   wherein the detection of protein-agent complex in step (c) indicates that the patient is likely susceptible to suffering from Parkinson's disease.

This invention also provides a method of assessing the likelihood that a patient is suffering from Parkinson's disease which comprises:
(a) obtaining a fluid sample from the subject;
(b) contacting the sample with an agent which forms a complex with a human dermcidin-1 (DCD-1) protein comprising consecutive amino acids having the sequence set forth in SEQ ID NO: 1, 2, or 4, or homolog thereof, under conditions permitting any such protein or homolog thereof present in the sample to complex with the agent; and
(c) detecting the presence of any protein-agent complex formed in step (b),
   wherein the detection of protein-agent complex in step (c) indicates that the patient is likely susceptible to suffering from Parkinson's disease.

This invention also provides a method of assessing the likelihood that a patient is suffering from Parkinson's disease which comprises:
(a) providing a solid support to which an agent which forms a complex with a human dermcidin-1 (DCD-1) protein comprising consecutive amino acids having the sequence set forth in SEQ ID NO: 1, 2, or 4, under conditions permitting any human DCD-1 protein present in the sample to complex with the agent is bound;
(b) contacting the solid support from (a) with a fluid sample from the subject;
(c) removing any of the human DCD-1 protein which is not bound to the solid support; and
(d) detecting the presence of protein bound to the solid support,
   wherein the detection of the human DCD-1 protein bound to the solid support in step (d) indicates that the patient is likely suffering from Parkinson's disease.

This invention provides a method of assessing the likelihood that a patient is suffering from Parkinson's disease which comprises:
(a) obtaining a fluid sample from the subject;
(b) contacting the sample -with an agent which forms a complex with a protein encoded by a nucleic acid comprising consecutive nucleotides having the sequence set forth in SEQ ID NO:3, or fragment of the protein, under conditions permitting any such protein or fragment thereof present in the sample to complex with the agent; and
(c) detecting the presence of any protein-agent complex formed in step (b),
   wherein the detection of protein-agent complex in step (c) indicates that the patient is likely suffering from Parkinson's disease.

This invention also provides a diagnostic kit which comprises a container comprising a solid support to which an agent which forms a complex with a human dermcidin protein comprising consecutive amino acids having the sequence set forth in SEQ ID NO: 1, 2, or 4 is bound, which agent is labeled with a detectable marker.

This invention further provides any of the instant methods, wherein the sample is cerebrospinal fluid or a derivative of cerebrospinal fluid. This invention further provides any of the instant methods, wherein the sample is blood or a derivative of blood. This invention further provides any of the instant methods, wherein the sample is serum, lymph or synovial fluid, or a derivative thereof, for example a centrifugate.

This invention further provides any of the instant methods, wherein the agent is an antibody. This invention further provides any of the instant methods, wherein the antibody is a monoclonal antibody or a polyclonal antibody. Such antibodies may be human, rat, rabbit, goat, or chicken derived, and may be synthesized by techniques well known and long-established in the art using the protein or protein fragment to be detected.

This invention further provides any of the instant methods, wherein the antibody is a labeled antibody and wherein the detecting of the presence of protein-agent complex is effected by detecting the label on the antibody. This invention further provides any of the instant methods, wherein the antibody is labeled with a radioisotope, a chromophore, a biomolecule, a fluorophore, a radiolabeled molecule, a dye, an affinity label, an antibody, biotin, streptavidin, a metabolite, a mass tag, or a dextran.

Detection may be performed using mass spectrometry, including SELDI technology, fluorimetry, radiometry, western blot assays, ELISA assays, other immunoassays, including agglutination assays wherein monoclonal antibodies directed against the dermcidin protein or fragment are attached to solid particles such as latex or polystryrene, or carbon in a carbon sol, and wherein presence of the protein or fragment in the sample elicits agglutination of the particles, or surface plasmon resonance wherein the antibodies are attached to a plasmon chip and binding of the protein or fragment to the antibodies elicits a change in surface plasmon resonance signal.

This invention further provides any of the instant methods, wherein the patient is suffering from a neurodegenerative disease.

This invention further provides any of the instant methods, further comprising determining the amount of complex formed and comparing such amount with a standard, wherein a greater amount of complex formed in step (b), or step (d) as appropriate, than in the standard indicates that the subject is likely suffering from Parkinson's disease.

This invention also provides a diagnostic kit which comprises a container comprising a solid support to which an agent which forms a complex with an human DCD-1 protein comprising consecutive amino acids having the sequence set forth in SEQ ID NO: 1, 2, or 4, or a homolog thereof, is bound, which agent is labeled with a detectable marker.

The methods of this invention may be performed with dermicidin precursor protein or with DCD-1 or immunoreactive fragments each thereof as the biomarker.

Early diagnosis of PD permits tailoring of therapies to PD, and helps avoid employing inappropriate therapies which may be employed in similarly presenting neurodegenerative disorders, such as other neurodegenerative movement disorders, progressive supranuclear palsy. Early diagnosis also permits earlier intervention and therapy and consequent improved prognosis. Employing further biological markers of PD in addition to the dermcidin markers may offer an even more precise diagnostic tool. High throughput screening technologies allow rapid diagnosis.

Therapies employing expression and secretion control of dermcidin forms to impede production of truncated/cleaved dermicidn may thus be useful in treating PD also. Technologies such as catalytic nucleic acids (DNAzymes and Ribozymes directed to the protein to be inhibited from expressing), RNAi, antisense directed to the mRNA encoding DCD and DCD precursor, for example directed against and hybridizable with SEQ ID NO:3 under stringent conditions, monoclonal antibodies directed to SEQ ID NOs. 1, 2, or 4, and small molecules may be employed. Therapy may be effected by in vivo or ex vivo gene therapy, for example standard techniques to silence DCD-1 in the subject. Therapy may be achieved by inducing expression of full length DCD or inhibiting DCD processing in the subject. All of the instant methods may be performed in animal models of Parkinson's disease, including rat and mouse models.

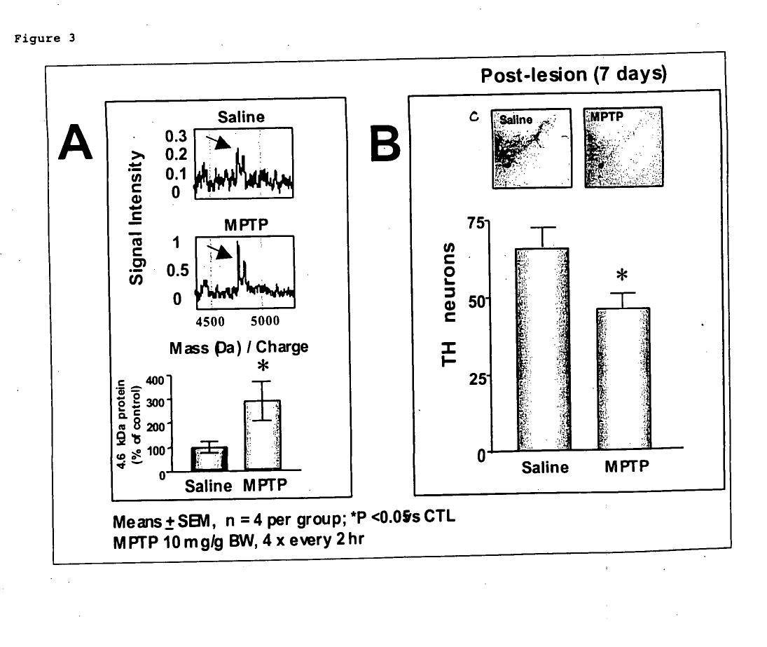

As used herein “a human dermcidin-1 (DCD-1)” means a polypeptide which has the same or substantially the same amino acid sequence as a naturally occurring human DCD-1, including specifically, SEQ ID NO:1. The term “a human DCD-1” thus encompasses naturally occurring human DCD-1 variants such as muteins, isoforms, polymorphisms, allelic variant, polypeptides encoded by an alternative splice form of a native human DCD-1 gene, and polypeptides encoded by a homolog of a native human DCD-1 gene. The peptide sequence of such variants can feature a deletion, addition, or substitution of one or more amino acids of a native human DCD-1 protein. The term encompasses the sequence of DCD-1 as set forth in the DCD sequence of FIG. 3c of Schittek et al., (2001) Nature Immunology 2(12): 1133-1137, and the DCD-1 peptide of dermcidin precursor protein GenBank Accession No. AF144011,

(SEQ ID NO:2)

MRFMTLLFLTALAGALVCAYDPEAASAPGSGNPCHEASAAQKENAGEDPG
LARQAPKPRKQRSSLLEKGLDGAKKAVGGLGKLGKDAVEDLESVGKGAVH
DVKDVLDSVL;

(SEQ ID NO:1)

SSLLEKGLDGAKKAVGGLGKLGKDAVEDLESVGKGAVHDVKDVLDSV,

Accession No. AAL18349; and fragments such as DAVEDLESVGK (SEQ ID NO:4).

DCD-1 protein fragments corresponding to one or more particular motifs and/or domains or to arbitrary sizes, for example, at least 5,10,15,20,25,30,35, or 45, amino acids in length are within the scope of the present invention. In one embodiment, the fragments are immunoreactive.

Conditions permitting agents, e.g. antibodies, to complex with DCD-1 proteins or fragments thereof, means conditions which allow e.g. an antibody to complex with, or bind to an epitope present on, the protein. Conditions allowing specific interaction mean conditions permitting the antibody, for example, binding to a detectably greater degree (e.g., at least 2-fold over background) than the antibody binds to substantially all other epitopes in a reaction mixture comprising the particular epitope. In the case of immunoassays, such ‘immunologically reactive’ conditions are dependent upon the format of the antibody binding reaction and typically are those utilized in immunoassay protocols. See Harlow and Lane, Antibodies, A Laboratory Manual, Cold Spring Harbor Publications, New York (1988), for a description of immunoassay formats and conditions. A variety of immunoassay formats may be used to detect antibodies reactive with a particular agent. For example, solid-phase ELISA immunoassays are routinely used to select monoclonal antibodies specifically immunoreactive with a protein. See Harlow and Lane, Antibodies, A Laboratory Manual, Cold Spring Harbor Publications, New York (1988), for a description of immunoassay formats and conditions that can be used to determine selective reactivity.

Sequence identity between variants is the similarity between two nucleic acid sequences, or two amino acid sequences is expressed in terms of the similarity between the sequences, otherwise referred to as sequence identity. Sequence identity is frequently measured in terms of percentage identity (or similarity or homlogy); the higher the percentage, the more similar the two sequences are. Homologs of the human DCD-1 proteins will possess a relatively high degree of sequence identity when aligned using standard methods.

Methods of alignment of sequences for comparison are well-known in the art. Various programs and alignment algorithms are described which present a detailed consideration of sequence alignment methods and homology calculations. Additionally, the NCBI Basic Local Alignment Search Tool (BLAST) (Altschul et al., 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI online site under the “BLAST” heading. A description of how to determine sequence identity using this program is available at the NCBI online site under the “BLAST overview” subheading.

Homologs of the disclosed human DCD-1 protein are typically characterized by possession of at least 70% sequence identity counted over the full length alignment with the disclosed amino acid sequence of either the human DCD-1 protein amino acid sequences using the NCBI Blast 2.0, gapped blastp set to default parameters. Proteins with even greater similarity to the reference sequences will show increasing percentage identities when assessed by this method, such as at least 75%, at least 80%, at least 90% or at least 95% sequence identity. When less than the entire sequence is being compared for sequence identity, homologs will typically possess at least 75% sequence identity over short windows of 10-20 amino acids, and may possess sequence identities of at least 85% or at least 90% or 95% depending on their similarity to the reference sequence. One of skill in the art will appreciate that these sequence identity ranges are provided for guidance only; it is entirely possible that strongly significant homologs could be obtained that fall outside of the ranges provided. The present invention provides not only the peptide homologs are described above, but also nucleic acid molecules that encode such homologs.

One indication that two nucleic acid sequences are substantially identical is that the polypeptide which the first nucleic acid encodes is immunologically cross reactive with the polypeptide encoded by the second nucleic acid. Another indication that two nucleic acid sequences are substantially identical is that the two molecules hybridize to each other under stringent conditions. Stringent conditions are sequence dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (1989). Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature of the probe to about 20° C. to 25° C. below the melting temperature) . One or more factors be may be varied to generate conditions of either low or high stringency different from, but equivalent to, the above listed conditions. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequence that all encode substantially the same protein.

This invention further provides a monoclonal antibody directed to SEQ ID NO:1, 2, or 4, or a fragment/homolog thereof.

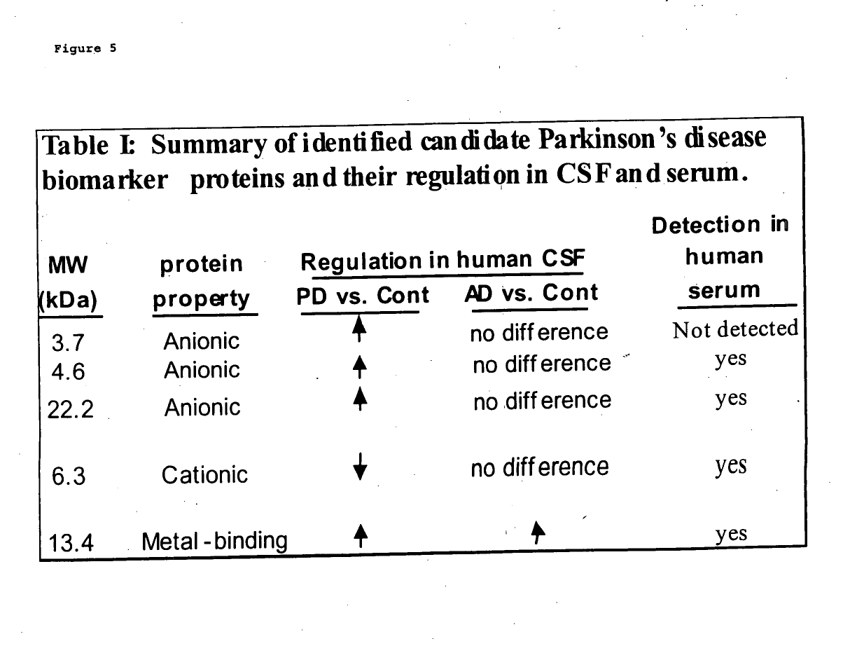

This invention further provides an animal model of Parkinson's disease, wherein the model expresses a processed form of DCD and/or other markers detailed in FIG. 5. In different embodiments the model may be a mouse model or a rat model.

The invention further provides a method of assessing the likelihood that a patient is suffering from Parkinson's disease which comprises the steps of the instant methods and determination of the presence of any or all of the markers detailed in FIG. 5, wherein the presence of two or more of the markers indicates that the patient is suffering from Parkinson's disease.

This invention will be better understood by reference to the Experimental Details which follow, but those skilled in the art will readily appreciate that the specific experiments detailed are only illustrative of the invention as described more fully in the claims which follow thereafter.

Experimental Details

First Series of Experiments

SELDI Proteomics: Implications in the Search of Biomarkers in Parkinson's Disease (PD)

We investigated using high-throughput proteomic studies to identify protein profiles for developing a rapid, sensitive and specific high-throughput diagnostic assay for PD. Although 2-D electrophoresis can effectively resolve a large number of proteins, its limited reproducibility renders it difficult for discovery/validation studies involving multiple cases and multiple disease stages. The advantage that SELDI ProteinChip technology has over 2-D electrophoresis is that the same chip platform used to identify the biomarker can also be used to develop a rapid, sensitive and high-throughput assay. Protein profiling using SELDI technology offers a novel means for PD diagnosis, and monitoring the PD clinical progression. Investigation of individual and composite fingerprint profiles of protein expression as a function of the various stages and sub-types of PD, may provide further criteria for identifying and tracking the onset and progression of clinical PD. The great advantage of utilizing a group of biomarkers is that we will not be constrained by the sensitivity or specificity of any single biomarker, which may be low or may vary with PD sub-type. In the studies using SELDI protein profiling as a tool for biomarkers discovery in PD, we found a combination of up-regulated and down-regulated proteins in CSF of cases characterized by PD. Overall, our evidence shows the usefulness of this systematic approach to identify potential novel molecular indexes whose content in biological fluids would specifically predict the onset and progression of PD.

Brain vs. Body Fluids: Implications in the Identification of Biomarker of PD Onset and Progression

Body fluids have the advantage, over the brain, of being more easily accessible for studying PD progression. Since procuring serum and, to some extent CSF, is a non-invasive procedure, they are more likely to be of clinical use. However, the identification of a biomarker in these samples requires that the protein be secreted at high enough levels to be identified. Moreover, circulating factors may be secreted by multiple cell types from multiple organs, and therefore, may lack specificity to a pathophysiological event occurring in the brain. For example, a complicating factor for studies of biomarker studies in serum is that albumin, transferrin and IgG, in general, are likely to be much more abundant than the potential biomarkers and may interfere with biomarker detection.

While the brain may still represent a key starting point in the search for novel biomarkers of PD, our SELDI studies support the usefulness of this technology to study variation in protein expression patterns in cerebrospinal fluid (CSF) and serum, during the transition from normal motor function to clinical PD. Our evidence is that the CSF content of Dermicidin (DCD), a recent described glycoprotein with multiple functions, is a novel potential PD biomarker. DCD is a 24 kDa sulfated glycoprotein encoded by a single gene known as DCD precursor protein 19-21, localized on a chromosome^{12 3} which encodes a secretory precursor protein of 11 kDa with two N- and two O-glycosylation sites²⁰. Although the specific biological function of DCD is not known, potential functions mediated by DCD precursor protein recently described include: 1) muscle protein degradation during cachexia^{19, 2}) pathogenesis of malignant melanoma^{21, 3}) abnormal glucose utilization²², 4) neuroprotection against oxidative stress^{2,23} and, 5) anti-microbial activity in sweat 3 and, most recently, growth and survival in breast cancer cells²⁴. We disclose transfection studies that indicate that DCD precursor protein expression in N2A neuroblastoma cells, may also neuroprotect against H₂O₂ mediated neurotoxicity, consistent with previous evidence showing that peptic fragment corresponding to aa 20-49 of DCD (known as YP30) may be neuroprotective^{2,23}. Our studies indicate that DCD in PD may reflect a compensatory response to oxidative stress conditions.

Feasibility Survey Studies in PD Biomarker Discovery

For these studies, CSF and serum samples from idiopathic PD cases were obtained through collaboration with the Parkinson Institute, Pao Alto (Dr. Di Monte, Collaborator). In view of the exploratory nature of these feasibility studies, strict exclusion criteria were used to avoid potential confounding conditions in the interpretation of the study. Patients were matched closely for clinical history by H&Y rating 5 (average 2-2.5), age at onset (64+6), drug history (e.g. L-DOPA), disease duration (5.8+3.2), lack of dyskinesia/on/off conditions); cases with intercurrent infection and other inflammatory conditions were also excluded. Neurologically controlled cases, generally the spouses of PD cases were age matched (59+9). In addition, to further assess whether changes in CSF protein expression profiles were specific to PD, or are non-specific index of generalized ongoing neurodegenerative events, we also explored the specificity of these changes by assessing their CSF content of age matched, probable AD cases based on mini mental (MMSE) score (provided through a previous collaboration with Dr. Harald Hampel (Ludwig-Maximilian University, Munich, Germany; MMSE score range 21-26²⁵ indicative of probable AD dementia; mean age=68+11; n=6). In further control studies, in collaboration with Dr. Merit Cudkowicz, Neurology Service, Harvard Medical School, we also explored the selectivity of PD changes in CSF in respect with changes in CSF of pure Amyotrophic Lateral Sclerosis (ALS) (excluding spinal-bulbar muscular atrophy). The studies using these pre-banked un-identified samples were approved by the IRB at Mount Sinai School of Medicine.

Identification of a Novel Processed Form of Proteolysis-Inducing Factor (DCD)—A Potential Novel Biomarker of PD.

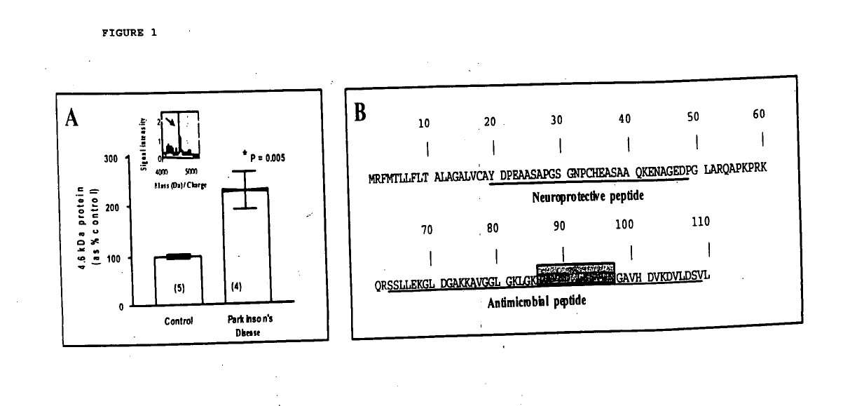

In ongoing liquid chromatography/mass spectrometry (LC-MS/MS) studies in our labs, we determined the amino acid (aa) sequence of an 11-aa residue proteolytic fragment (DAVEDLESVGK) (SEQ ID NO:4) cleaved from a 4.6 kDa anionic protein species (see FIG. 1A and FIG. 1B, highlighted by a box) whose content was selectively elevated in CSF of PD (n=5) vs. neurological control cases (n=4) by SELDI mass spectrometry (FIG. 1A).

CSF was collected by lumbar puncture (n=4 PD and 5 neurological control cases). In general for these studies, soon after collection, CSF samples were centrifuged (13,000×g) at 4° C., aliquoted and stored at −80° C. (for shipment on dry ice); once thawed, samples were stored at 4° C. For SELDI studies 4 μg of CSF proteins from each case was analyzed by SELDI technology using the SAX protein chip; only anionic (basic) proteins are analyzed using this chip. Resultant spectra from all cases are simultaneously normalized based on the total signals detected in individual spectra, in order to correct for minor differences in sample loading. A clustering program (Ciphergen) was used to analyze the content distribution of individual proteins for each individual case as a function of PD and neurological control grouping. Panel A shows the content of the 4.6 kDa cationic protein in CSF from PD and control cases; data are expressed as mean+SEM, number in parenthesis indicates the number of cases analyzed for each specified group; *P<0.05. The 4.6 kDa anionic protein was purified using K30 spun-column size chromatography, following manufacturer's instructions (Ciphergen, Fremont, Calif.). In panel A (inset), an aliquot of purified protein fraction was assessed by SELDI protein chip to confirm recovery of the 4.6 molecular mass (DA)/charge peak. In panel B, LC-MS/MS sequence identification of an 11-aa residue proteolytic fragment (highlighted in box) matching aa residues 86-96 of the human DCD precursor protein sequence (match derived using mass based database search software).

Briefly, the 4.6 kDa target protein species was purified by K30 spun-chromatography followed by SDS-PAGE, tryptic digested and injected in a LC-MS/MS, which generated mass spectra (based on mass Da/charge ratio) reflecting the sequence of the fragmented peptide. Based on LC-MS/MS spectra information, a protein sequence was assigned by database search using a specific search engine for identifying proteins from MS/MS spectra information (Sonar form ProteoMetrics Canada Ltd., Winnipeg, Manitoba). As shown in FIG. 1B, the sequenced 11-aa residue PD biomarker matched aa residues 86-96 of the human (h)DCD precursor protein^{2,19-21} also known as dermcidin (DCD)³ (FIG. 1B). The function of DCD precursor protein is not well understood, however it is known that DCD is processed into multiple (secreted and intracellular) polypeptides with varying biological activities and functions. For example, the peptic fragment corresponding to aa 20-49 (underlined in FIG. 1B) was recently described as an H₂O₂-responsive gene product in neuronal cells, suggesting a role for this peptide in oxidative stress responses^{2,23}. Additionally, recent evidence also indicated that aa 63-109 encodes a novel peptide defined as DCD-1 (also underlined in FIG. 1B) with potent anti-microbial activities in sweat 3. Finally, aa 19-39 sequence appears to encode for a novel peptide involved in muscle protein degradation^{19,20}. Within the hDCD sequence, the aa residues 1-19 encode a signal peptide sequence while aa 20-110 correspond to the mature DCD precursor protein.

In addition to DCD, in SELDI studies, we chromatographically identified 4 other novel protein biomarkers (2 anionic, 1 cationic protein species and 1-metal binding protein species) whose content, is altered in the CSF of PD cases, relative to age matched neurological controls (see FIG. 5). Most importantly in preliminary studies we found that, with the exception of a 3.7 kDa anionic protein, all of our candidate CSF PD biomarker proteins, including DCD, are detectable in serum with a high degree of resolution. This confirmation supports our additional aim to explore the regulation of novel PD biomarker proteins in the serum.

DCD-1 Immunoreactive Material as a Potential Novel PD Biomarker.

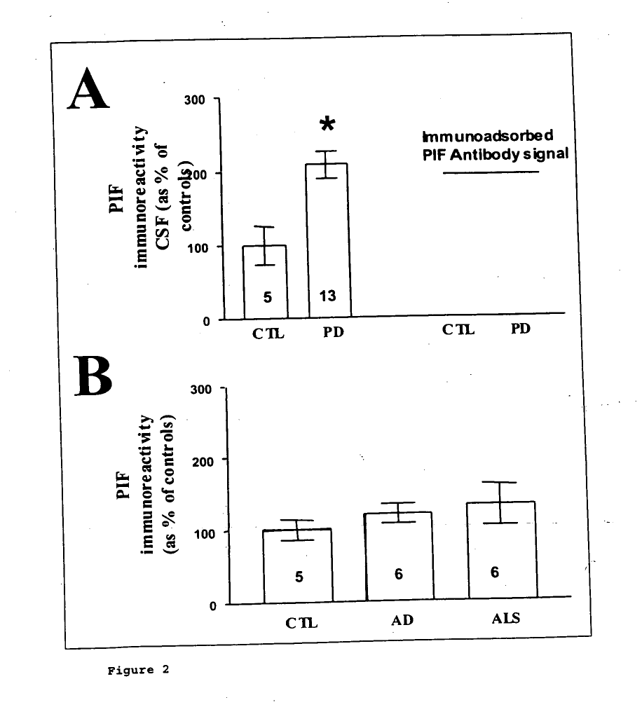

Based on the evidence that DCD may be a novel PD biomarker in CSF, we generated a quantitative ELISA assay to validate our mass spectrometry SELDI evidence. We generated a polyclonal DCD antibody raised against a synthetic DCD peptides spanning aa 95-109 of hDCD precursor protein (FIG. 1B) (rabbit anti-human DCD95-109). Using this antibody, constructed a DCD precursor protein ELISA assay in our labs based on procedure described by Segawa et al, 2003²⁶. As shown in FIG. 2A, we found that DCD immunoreactive material content in CSF was selectively elevated in PD cases (n=13), relative to age matched CSF from neurologically normal control (NC) cases (n=5) (PD vs. NC; P<0.05; t test) . In control studies we found that immunoadsorption of the rabbit anti-human DCD95-109 with synthetic DCD peptides spanning the aa sequence 95-109, completely abolished DCD immunoreactivity, supporting the specificity of DCD detection by our ELISA assay (FIG. 2a). DCD immunodetection in our ELISA assay system was linear within 100-800 fold dilution of CSF (not shown). Most importantly, in control ELISA assays we found that the DCD immunoreactive material in the CSF of PD cases was highly specific, with no detectable elevation of DCD immunoreactive material found in the CSF of cases characterized by probable AD (n=6) or ALS (n=6), relative to neurologically normal control cases. Moreover, there was no significant difference in mean age (ANOVA, p=0.40) among neurological controls, PD or ALS groups.

In panel A of FIG. 2, detection of DCD immunoreactive material by ELISA in CSF from PD and neurologically normal or neurodegenerative control cases; for antibody immunoadsorption, the rabbit anti-human DCD95-109 was pre-incubated at room temperature with 1000 fold higher concentrations of a synthetic DCD peptide (aa 95-109) (based on molar ratio), for 24 hr. In panel B, parallel studies assessed DCD immunoreactive material by ELISA in CSF of probable AD and ALS samples. In this study microtiter wells were coated with antigen (0.5 μl CSF diluted 200-fold) dissolved in 100 μl of 50 mM carbonate buffer, pH 9.6, and incubated at 4° C., overnight. All subsequent incubations were made in a volume of 50 μl/well. The plates were blocked with dilution buffer (10 mM Tris-HCl buffer, pH 7.4, containing 0.05% Tween 20, 0.5 M NaCl and 5% skimmed milk) followed by incubation with the primary rabbit-anti-human (DCD 95-109 ) antibody for 1 h at room temperature. The plates were incubated with biotin-conjugated anti-rabbit IgG in dilution buffer for 1 h and then incubated with horseradish peroxidase-conjugated streptavidin (Amersham Biosciences) for 15 min. Color was developed with 3,3′,5,5′-tetramethyl-benzidine (0.4 mg/ml) in 0.05 M citric acid phosphate buffer, pH 5.0, containing H₂O₂ (0.006%). Absorbance at 630 nm was measured with Coulter microplate reader. Data are shown as Mean+SEM (in duplicates) . *P<0.05 vs. neurological controls; abbreviations: CTL, neurologically normal control, AD, Alzheimer's disease; PD, Parkinson's disease.

Characterization of Protein Biomarkers in a Mouse Model of PD-Type Neurodegeneration.

Based on the data from studies aiming at identifying potential novel PD biomarkers, in ongoing studies we initiated a series of pre-clinical investigations to further explore the predictability of potential PD biomarkers in PD models in vivo.

MPTP-Mediated Substantia Nigra DAergic Neurotoxicity in Mice is Associated with Elevation of a 4.6 kDa DCD Like Protein Species in the CSF.

In this study SN DAergic lesions in male C57B6/SJ mice were obtained by i.p. MPTP injection (four consecutive injections at 2 hr intervals; 20 mg/kg/body weight (BW) per injection 27); saline injected mice were used as a control (n=4 per group). Four days following MPTP (or saline) injection, mice were anesthetized using a combination of ketamine (50 mg/kg BW) and xylazine (5 mg/kg BW), CSF was then collected from cisterna magna, as previously described²⁸. Immediately after CSF collection (4 days post-lesioning), mice were sacrificed by cervical dislocation and the brain was collected, preserved under cryoprotection and stored at −80° C. 1 μl of CSF from each animal was analyzed by SELDI technology as described in FIG. 1. In panel 3A, detection and quantification of 4.6 kDa DCD-like protein induction in the CSF of MPTP lesioned mice, as indicated. In panel 3B, number of thyroxine hydroxylase (TH)-immunoreactive DAergic neurons in the SN counted stereologically, with data are expressed as mean+SEM for MPTP and saline groups. In panels A and B, n=4 per group; *P<0.05. Panel 3C (inset), representative photomicrograph identifying the distribution of TH-immunoreactive neurons in the SN region in response to MPTP- or saline-injection (see FIG. 3).

In encouraging SELDI high-throughput mass spectrometry studies we recently chromatographically identified five novel anionic biomarker protein species whose content was elevated or reduced in the CSF following MPTP lesions in mouse, which models PD SN neurodegeneration, relative to saline injected mice (not shown). Among the five candidates protein species whose content was altered in the CSF of MPTP-lesioned mice there was a 4.6 kDa anionic protein with biophysical properties similar to human DCD (based on mass Da/charge and chemical protein binding at pH 8 in a cationic environment), which was induced in the mouse CSF in response to MPTP lesions (4 days post-lesioning). Most importantly, we found that the elevation of the 4.6 kD anionic protein species following MPTP lesions coincided with approximately 25% loss of tyrosine hydroxylase (TH) immunopositive neurons, assessed stereologically (FIG. 3B). In collaboration with Dr. Wang (Collaborator, Proteomics Laboratory, Department of Genetics, Mount Sinai School of Medicine), we are presently sequencing this 4.6 kDa DCD-like protein species from mouse CSF to confirm its identity.

Studies Supporting the use of Magnetic Resonance Microscopy (MRM) to Volumetrically Assess Atrophic Changes in the Substantia Nigral Region of Midbrain for Correlation with CSF Biomarker Expression in PD-Type Neurodegeneration.

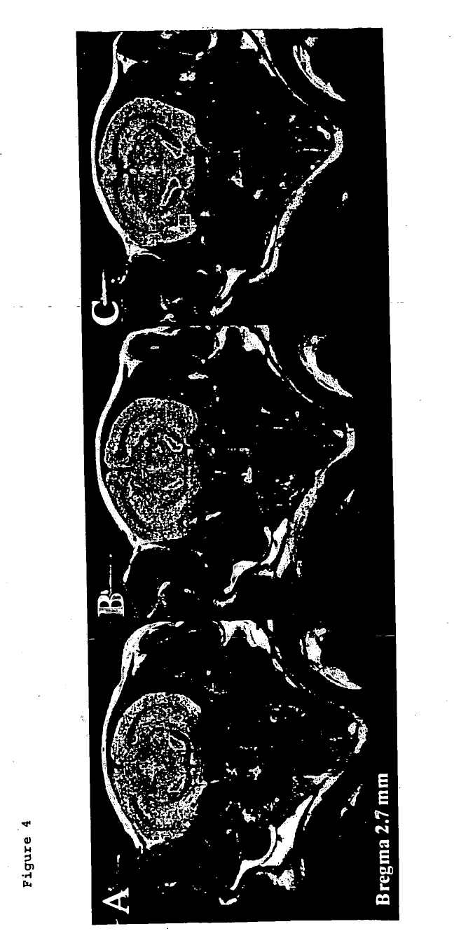

The goal of this study was to develop a non-invasive methodology for functionally assessing the predicative role of novel protein biomarkers in CSF (and serum) for experimental MPTP SN neurodegeneration in pre-clinical models of PD (Aim 4) using MRM imaging technology in collaboration with Dr. Cheuk Tang (Collaborator, Imaging Sciences Laboratory, Dept. Neuroradiology) and Dr. Patrick Hof (Collaborator, Advance Imaging Program, Center for Neurobiology), of Mount Sinai Medical School. The studies are based on the principle that increased iron content found in SN of PD and MPTP models of PD²⁹⁻³¹ may be an index of SN neurodegeneration, which can be monitored by MRM^{32,33}, longitudinally. Given that MRM measurement is highly sensitive to iron content in SN dopaminergic cells, we hypothesized that MRM may offer a means for non-invasive evaluation of SN neurodegeneration. In exciting ongoing studies, using a 9.4 Tesla micro MRM imager (Bruker Instrument) we collected matched consecutive rostro-caudal T2-weighted in vivo MRM images of the whole brain from the same mouse, prior to and four-days after SN MPTP lesion. We found that averaged T2-weighted values measured from three consecutive rostro-caudal slices of SN of MPTP lesioned animals (see SN tracing in FIG. 4) were decreased by approximately 25% relative to those collected from the SN of the identical brains pre-lesion (n=2). As predicted, this observation of hypo-intense MRM measurements in the SN post-lesioning is consistent with an elevation in iron content in the degenerating nigrostriatal DAergic neurons.

High-Resolution in vivo MRM of Whole Mouse Brain.

In this study SN DAergic lesions in male C57B6/SJ mice were obtained by IP MPTP injection (four consecutive injections at 2 hr intervals; 20 mg/kg/BW per injection. In panels A, B and C of FIG. 4 high-resolution 500 μm-thick in vivo MRM images along the rostra-caudal axis of one adult mouse brain using a 9.4 Tesla micro MRM imager (Bruker Instruments), 4 days after MPTP lesion. For imaging, individual mouse (which was also imaged before MPTP lesion) was maintained under general anesthesia using 1.5% isofluorane and carefully monitored for vital signals (temperature, respiration etc.). A 30 mm (I.D.) RF birdcage resonator was used in conjunction with a gradient insert of 100 G/cm; this coil encompasses the animal cradle specially designed for mouse imaging which includes a provision for inhalation anesthesia and respiratory and cardiac monitoring. In panels 4A, B and C, three consecutive optical sections at three rostro-caudal extents of the SN were generated from. the same mouse two weeks after MPTP lesions. The dorsal, lateral and medial landmark borders used for SN tracings (bilateral green oval lines) are respectively, peripeduncular nucleus, cerebral peduncle, and substantia nigra (inclusive) . In 4C, the bilateral green square tracing identifies the regions randomly selected for T2 signal assessment of the temporal cortex for background normalization. In this study (N=2 animals), the average SN normalized SN T2 density (calculated as SN T2 density/temporal T2 density) pre- and post-MPTP lesioning are, respectively, 0.9015+0.03 and 0.6904+0.05, corresponding to approximately 25% decrease in SN T2 density measurement in response to MPTP lesioning. In panel 4A, distance from Bregma—2.7 mm.

Collectively, the preliminary studies demonstrate that MRM technology can be used in vivo to evaluate MPTP-mediated SN neurodegeneration, strongly supporting the possibility of combining non-invasive MRM and high-throughput SELDI biomarker discovery technologies in longitudinal assessment of SN neurodegeneration.

Further Characterization of Other Potential Novel PD Biomarkers

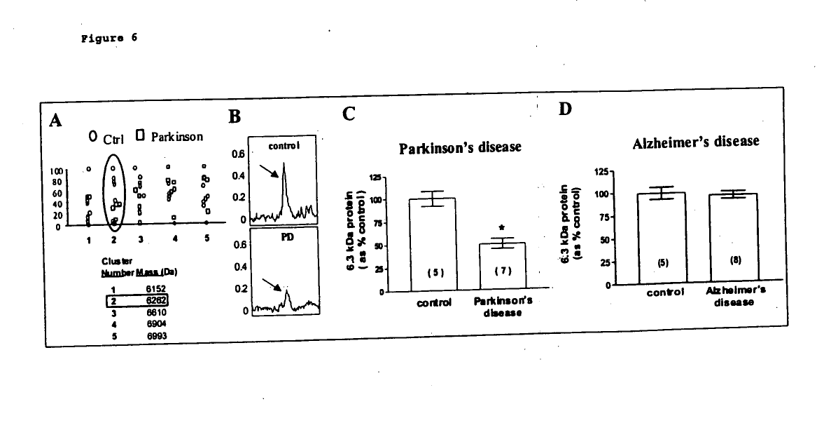

In ongoing SELDI high-throughput mass spectrometry, we have chromato-graphically identified other novel protein biomarkers (2 anionic, 1 cationic protein species and 1-metal binding protein species) whose content, in addition to DCD (anionic 4.6 kDa protein described above), was altered in the CSF of PD cases, relative to age matched neurological control cases (FIG. 5). These encouraging findings support our Aim 3 to continue to explore the regulation of potential novel protein biomarkers in the serum and CSF of PD cases. As shown in FIG. 6. we identified a 6.3 kDa cationic protein whose level of expression was found to be selectively decreased in the CSF of PD cases, compared to neurologically normal controls (FIG. 6, A-C) . (Based on this information, we next assessed whether the “down-regulation” of this 6.3 kDa cationic protein in the CSF was selective for PD versus a non-specific index of neurodegenerative conditions, as performed in our studies for DCD. We found that levels of the 6.3 kDa species in CSF of probable AD cases did not differ from controls (FIG. 6 D).

Investigation of a Cationic Protein Species as Novel Biomarker of PD.

For these studies CSF was collected by lumbar puncture (n=7 PD and =5 neurological control cases. 4 μg of CSF protein from each case was analyzed by SELDI technology using the WCX protein chip—only cationic (acidic) proteins are analyzed using this chip. Resultant spectra from all cases were simultaneously normalized based on the total signal detected in individual spectra; in order to correct for minor differences in sample loading. A clustering program (Ciphergen) was used to analyze individual proteins for each individual case as a function of PD and neurological control grouping. FIG. 6, panel A shows a molecular weight frequency scatter graph indicating the quantitative distributions of individual cationic proteins for each of the cases analyzed. PD and control cases are shown in blue squares and red circles, respectively. Molecular sizes for each of the protein clusters are indicated at the bottom of the panel. Cluster #2 (enclosed in the circle) shows the level of expression of the 6.3 kDa cationic protein in PD and control cases. Panel 6B shows representative SELDI retention maps where the “peaks” represent individual detected proteins, and the area under the peak represents the signal intensity. Red arrows indicate the 6.3 kDa cationic protein. Panel 6C shows the content of the 6.3 kDa cationic protein in CSF from PD and control cases. Panel D shows the content of the 6.3 kDa cationic protein in CSF of neuropathologically confirmed AD compared to cognitive normal control cases. In both panels 6C and 6D, data are expressed as mean +SEM, number in parenthesis indicates the number of cases analyzed for each specified group; *P<0.05.

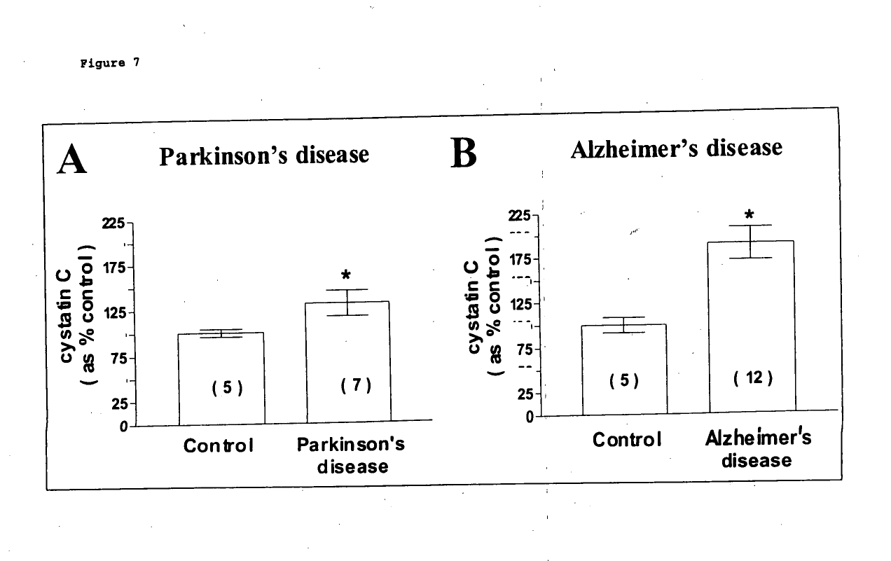

Based on this evidence, we examined the expression of this potential novel 6.3 kDa biomarker in the CSF of probable AD cases, compared to neurologically (cognitively)—normal control cases (FIG. 6D). Excitingly, our observations demonstrated that CSF of AD cases contains only “baseline” levels of this 6.3 kDa cationic protein, identical to that of cognitively normal controls. In addition to the 6.3 kDa cationic protein, in more recent studies we also identified a 13.4 kDa metal-binding protein whose level of expression was found to be increased in the CSF of PD cases compared, to neurological control cases (FIG. 7A). LC-MS/MS sequencing studies showed this 13.4 kDa metal binding protein to be the immunomodulatory molecule cystatin C, which we also found to be elevated in CSF of probable AD cases, see FIG. 7B.

Elevated Expression of Cystatin C in the CSF of PD and Probable AD.

In this study, 4 μg of CSF protein from each case was analyzed by SELDI technology using the IMAC-Cu++ protein chip—only metal-binding proteins are analyzed using this chip. See FIG. 7. CSF collection, data collection and analysis of normalized spectra are essentially as described in FIG. 1 legend. Panel 7A, content of cystatin C in CSF from PD and control cases. Panel 7B, content of cystatin C in CSF from probable AD compared to cognitively normal neurological control cases. In both panels 7A and B, data are expressed as mean+SEM, values in parentheses indicate the number of cases analyzed for each specified group; *P<0.05.

In addition to the aforementioned 6.3 kDa cationic protein and cystatin C, we also identified 3 novel anionic proteins (using the SAX protein chip for preferential analysis of basic proteins) whose level of expression was found to be increased in the CSF of PD cases, compared to neurological control cases. The molecular sizes and properties of these candidate biomarker proteins are summarized in FIG. 5. In view of our evidence that the 13.4 kDa metal-binding protein cystatin C is also detected in the serum of probable AD cases, using SELDI technology we next explored whether expression of any of the other novel biomarkers, found to be regulated in the CSF of PD, were also detectable in the serum. In preliminary observations, we found that, with the exception of the 3.7 kDa anionic protein, all of our candidate CSF PD biomarker proteins were detectable in serum with a high degree of resolution.

Investigation of the Potential Role of DCD Precursor Protein under Conditions of Oxidative Stress

We characterized potential mechanisms through which DCD precursor protein may influence PD.

DCD Receptor Binding in Midbrain SN Neurons

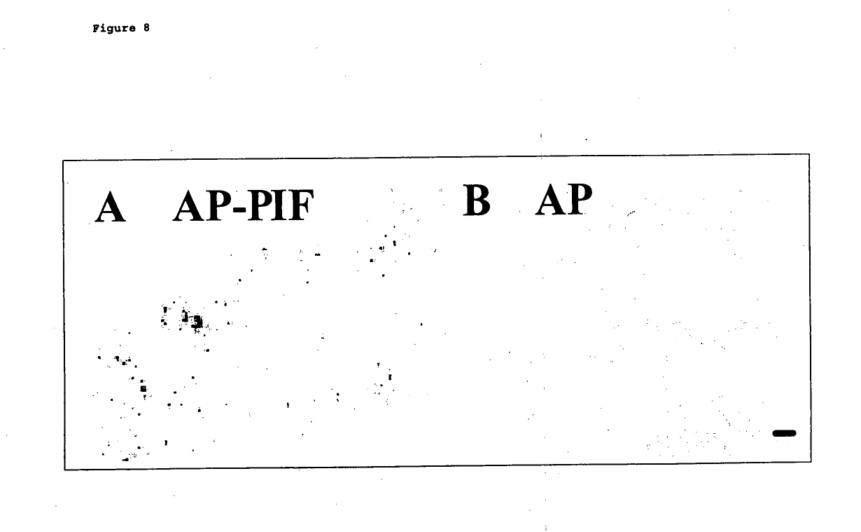

In view of the evidence that DCD (also known as Dermcidin^{3,20}) is a functionally secreted glycoprotein^{19,20}, and the recent evidence that its function could by mediated through binding to a cell-surface receptor²⁴, we initiated a series of studies to explore the distribution of DCD receptor in the brain of human neurological control cases. For these studies, in collaboration with Dr. Polyak (Collaborator), we generated an N-terminal alkaline phosphates (AP) C-terminal DCD precursor protein fusion protein as a ligand and used in receptor binding assays in situ (Porter et al., 2003²⁴) As shown in FIG. 8A, we found that in human brain high-intensity AP-DCD receptor binding was primarily localized to midbrain SN compacta neurons (based on distribution and size of labeled cells), relative to control AP background signal on adjacent tissue sections (FIG. 8B), consistent with a potential autocrine/paracrine mechanism of DCD action in SN compact neurons. Finally, in ongoing studies surveying the regional distribution of DCD binding in the brain of neurological control cases, we found that besides the midbrain SN compacta region, DCD receptor binding was also detectable in neurons of the locus ceruleus, pons, and lateral hypothalamic nuclei (not shown), consistent with previous finding (Porter et al., 2003²⁴).

In Situ Staining for DCD Receptor in Normal Human Midbrain SN Tissue.

In this study, 10 μm semi-adjacent cryo-sections of a normal brain specimen encompassing the midbrain SN region were incubated with AP-DCD fusion protein or AP control protein. See FIG. 8. Panel A: purple staining detects interaction between AP-DCD with a putative DCD receptor. Panel B: Faint background brownish coloring of neurons of the SN in the control AP sections is due to natural pigment (melanin) present in these cells. Abbreviations: AP-DCD, alkaline phosphatase-proteolysis inducing factor fusion protein, AP, alkaline phosphatase. Length bar, 100 μm.

Collectively, our evidence suggests that DCD may neuroprotect in vitro in response to H₂O₂ (FIG. 7) and that DCD is elevated in CSF of PD but not AD and ALS (FIG. 2) (and possibly in CSF in responses to MPTP lesions in mouse, FIG. 3) combined with the evidence showing DCD receptors in SN compacta neurons (FIG. 8), strongly support the possibility that DCD might play a neuroprotective role in DAergic neurons in PD.

The DCD-1 Proteolytic Fragment Promotes Dopaminergic Toxicity

Based on the observation that DCD-1 is the potential form of DCD that might predict clinical PD we decided to generate human recombinant DCD-1 and test the role of DCD-1 in experimental models of PD neurotoxicity. For generation of human recombinant DCD-1 (aa 63-109; Mol.wt. 4.7 kDa), the protein was expressed and purified using IMPACT-TWIN (Intein Mediated Purification with an Affinity Chitin-binding Tag-Two Intein) system from New England Biolabs, Inc., MA, according to manufacturer's recommendations. The IMPACT-TWIN system allows a target protein to be sandwiched between two self-cleaving inteins. Chitin binding domains present on both inteins allow the affinity purification of the precursor on a chitin resin, followed by pH and temperature dependent cleavage of intein releasing the target protein. Thus, in brief, primers were designed with 5′-SapI and 3′-PstI restriction sites for the amplification of DCD-1 DNA fragment spanning 247-387 nt of DCD human cDNA (Gene Bank Accession number NM_{—}053283), immediately followed by a stop codon. DCD-1 DNA fragment was amplified by PCR using human skin cDNA. The PCR amplified 150 bp fragment was restriction digested and cloned into TWIN1 vector. The nucleotide sequence of the vector construct was verified. E. coli. B cells (ER2566, New England BioLabs, Inc., MA) were transformed using vector construct and cells bearing the plasmid were grown at 30° C. in LB medium containing 100 μg/mL ampicillin to an A600 of 0.6-0.9. Expression of the fusion protein was induced by 0.3mM isopropyl □-D-thiogalactoside (IPTG) at 15° C. for 18h. The cells were collected by centrifugation and resuspended in ice-cold binding buffer (20 mM Hepes, pH 8.5, 0.5M NaCl, 1 mM EDTA). Then cells were broken by sonication at 4° C. After centrifugation clear extract was applied to a Chitin beads (New England Biolabs, Inc., MA) column pre-equilibrated with binding buffer at 4° C. The column was washed at 4° C. with 10 column volumes of binding buffer containing 0.1% Triton X-100, followed by 10 column volumes of binding buffer without detergent. Finally the column was equilibrated with 3 column volumes of cold cleavage/elution buffer (20 mM Hepes, pH 6, 0.5M NaCl, 1 mM EDTA). The column was incubated at room temperature for 16h with gentle rocking to induce cleavage of target protein from the fusion protein. DCD-1 was then eluted with elution buffer at room temperature. Pre-column and post-column fractions were analyzed by Coomassie Blue staining of SDS-PAGE and SELDI mass spectrometry. The First few elution fractions containing DCD-1 protein were dialyzed against 10 mM Na-phosphate pH 7, and stored at −20° C. Approximately 0.25 mg DCD-1 was obtained.

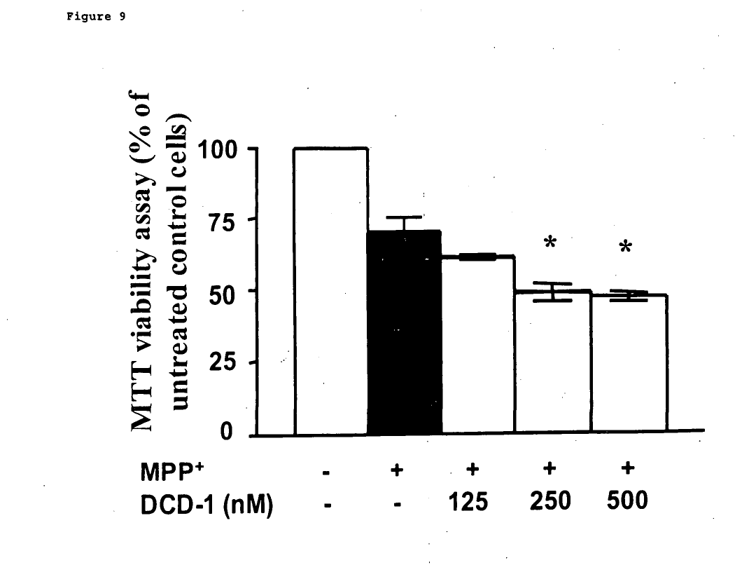

Next, using purified DCD-1, we tested its role on MPP+ mediated toxicity, that models select aspect of PD neurotoxicity, and found that DCD-1 potentiates MPP+ mediated death in SY5Y neuroblastoma catecholaminergic cells in a dose dependent fashion (FIG. 9). The PD biomarker DCD-1 promotes neurotoxicity in SY5Y cells in a dose dependent manner. In this study cell toxicity in SY5Y cells was assessed 24 hr after exposure to MPP+ using methylthiazoletetrazolium [3 (4,5-dimethylthiazol-2-yl)-2,5-diphenyltetrazolium bromide] (MTT) calorimetric assay, (an index or altered energy metabolism). Values represent Means+SEM of determinations from 2 separate cultures, n=3 cultures chambers per group * P<0.05 between MPP+ and MPP+ and DCD-1 treatment (see FIG. 9).

Materials and Methods

Surface-enhanced laser desorption ionization (SELDI) proteomic technology: SELDI is a system that enables rapid protein profiling, identification and characterization from crude biological samples. In particular, this system uses ProteinChip Arrays that contain chemically (cationic, anionic, hydrophobic, hydrophobic, etc) or biochemically (antibody, receptor, DNA, etc) treated surfaces¹⁴. Crude biological extracts (e.g. brain tissue extract, CSF, serum) are applied onto the ProteinChip Arrays, which selectively capture subclasses of proteins with specific physical or biochemical characteristics—based on interactions of proteins with the selected chip surface. The molecular size (MW) as well as the quantity of individual proteins absorbed on each chip is then directly assessed by a “time of flight”-mass spectrometer¹⁴. This generates aquantitative protein mass profile of the proteins bound to each of the ProteinChip Array surfaces. While control samples are always run in parallel with the experimental samples (CSF or serum samples from control and PD cases as disclosed here), the resulting profiles can be compared directly using a number of integrated software features that highlight relevant changes in the pattern of underlying protein expression. SELDI technology is highly sensitive (the lower limit of detection is 10 fmoles)¹⁵, quantitative, and highly reproducible. As such, SELDI technology, as used in our laboratories^{1,16} is highly amenable to high-throughput proteomic procedures, and presents a viable platform for biomarker discovery and validation. For protein identification purposes, the same chip platform used for biomarker discovery can easily be incorporated into strategies facilitating rapid target protein purification. Since purified protein can be digested “on-chip” with proteases, the same chip platform will be useful for generating peptide maps, which can be compared to a peptide database for protein identification. In addition, amino acid sequencing of either purified protein or peptic fragments can easily be used to derive the identity of the target species. SELDI technology offers advantages beyond cDNA microarray technologies as previously suggested for gene discovery studies applied to Alzheimer's disease^{1,17,18} and we disclose her applying that to PD.

Second Series of Experiments

We found that steady-state levels of proteolytic inducing factor (PIF) (Dermicidin) protein species is selectively elevated in the cerebral spinal fluid (CSF) in untreated Parkinson's disease (PD) with 81% sensitivity and 100% specificity. PIF is encoded by a single gene which is translated into a secretory precursor protein with multiple functions including neuronal responses to oxidative stress. Based on this evidence we continued to explore the potential role of PIF in PD pathophysiology and found that exposure of rat mesencephalic dopaminergic (DAergic) neuron cultures to human recombinant (hr)PIF promoted 1-Methyl-4-phenylpyridinium ion (MPP⁺) mediated neurotoxicity that was coincidental with elevated levels of soluble oligomeric α-synuclein protofibrils. This evidence is of high interest especially in view of ongoing studies in the lab showing that high affinity PIF cell surface receptors are primarily localized in brain regions undergoing degeneration in PD (e.g., midbrain DAergic neurons).

Mechanism through Which PIF Promotes DAergic Neurodegeneration in Substantia Nigra (SN).

We demonstrated that exogenous application of hrPIF to rat mesencephalic DAergic neuron cultures promoted a ˜2 fold potentiation of MPP⁺-induced neurotoxicity in an autocrine/paracrine manner which correlated with a commensurable elevation of soluble oligomeric α-synuclein protofibrils. We hypothesized that PIF may promote DAergic neurodegeneration by inducing the accumulation of soluble α-synuclein protofibrils, which has been recently demonstrated to be neurotoxic¹. Further supporting our hypothesis is the evidence that elevation of soluble oligomeric α-synuclein protofibrils precedes the formation of insoluble □-synuclein inclusions found in PD brain².

Characterization the Human Neuronal PIF Receptor using Expression Cloning Techniques.

Receptor binding studies in our lab have shown PIF receptor binding activity in SH-SY5Y cells, and have identified a single population of high affinity receptors using Scatchard analysis. Based on this evidence, we developed a COS expression cloning strategy to clone the human neuronal PIF receptor from SH-SY5Y neuroblastoma cells. This strategy is based on the expression of SH-SY5Y cDNA clones by transfection into COS cells. Expression of cell surface PIF receptor among transfected COS cells can be identified by screening for the capability to bind the alkaline phospatase (AP)-PIF fusion protein ligand. This permits identification of cDNA clone(s) encoding the human neuronal PIF cell surface receptor.

Identification of PIF and its Implications as a Novel Biomarker of PD.

We recently found that PIF levels in CSF were altered in non-treated PD suggesting that PIF is a novel potential PD biomarker. PIF is encoded by a single gene known as PIF precursor protein³⁻⁵, localized on chromosome 12 and encodes a secretory precursor protein of ˜11 kDa with multiple N- and O-glycosylation sites.⁵ Although the specific biological function of PIF is not known, recent evidence indicates that PIF (or prolytically processed forms of PIF) is involved in oxidative stress^{6,7}. Our new evidence showing that PIF promotes soluble oligomeric α-synuclein protofibril generation is relevant to PD, especially in consideration of in situ receptor binding-assay evidence showing that in the human brain PIFreceptor binding activity is primarily localized to midbrain SN DAergic neurons. These results suggest a novel function for PIF and/or its proteolytically processed forms in midbrain DAergic neurons.

PIF and its Potential Role in α-Synuclein-Mediated PD Pathophisiology.

The role of α-synuclein in the pathophysiological process of PD is far from obvious, but a prerequisite of α-synuclein neuropathy is its oligomerization into soluble protofibrils⁸ followed by coalescence into insoluble fibrils which are composed of α-sheets and amyloid-like filaments⁹. This phenomenon appears to precede aggregation into insoluble fibrillar structures and inclusions which accumulates into PD Lewy bodies (LBs). Thus, preventative inhibition of PIF-mediated promotion of soluble oligomeric α-synuclein protofibrils may provide a novel therapeutic approach to slow PD neuropathology. However, a puzzling aspect of α-synuclein-mediated neurotoxicity and LB formation is the preferential and selective neurodegeneration of DAergic neurons of the SN since α-synuclein is ubiquitously expressed at high levels in virtually all brain regions¹⁰. This suggests that the increase in intracellular concentrations of α-synuclein is not the sole factor that alters cell fate. For example, among the factors shown to affect cellular functions reciprocally with α-synuclein are ROS producing metabolic pathways and eventually mitochondrial dysfunction¹¹. Thus, the results disclosed here showing that 1) PIF mRNA expression is promoted in DAergic neurons in response to MPP⁺-induced neurotoxic injury correlated with elevated levels of soluble α-synuclein protofibrils and that 2) PIF receptor binding activity in the brain is primarily localized to DAergic regions (among other regions), is consistent with the hypothesis that PIF may represent a novel “factor” dictating the regionality of α-synuclein mediated neurotoxicity and possibly LB formation in PD.

PIF is Selectively Elevated in the CSF of PD

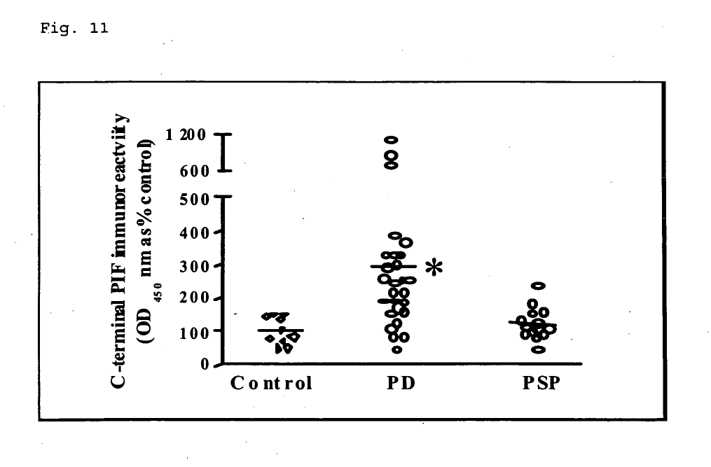

In ongoing SELDI-MS studies (a high-throughput system that enables rapid protein profiling of biological fluids), we identified a series of novel protein biomarkers whose content was altered in the CSF of idiopathic PD cases. Among others, we identified a 4.6 kDa protein species whose content was selectively elevated in the CSF of PD relative to neurologically normal control cases. Peptide sequence analysis identified this 4.6 kDa protein species to be a proteolytic fragment of the PIF precursor protein. Based on this evidence, we designed specific sandwich ELISA assays and confirmed the elevation of PIF steady-state levels in the CSF of a larger cohort of non-medicated PD-cases (from DATATOP collection) relative to neurologically normal control cases. As shown in FIG. 1A, consistent with the SELDI-MS data, we found that PIF protein content in the CSF of non-medicated PD (n=27) was selectively elevated relative to the PIF content in the CSF of neurologically normal control cases (n=11) (PD vs. NC; P<0.05; t-test). The increased steady-state levels of PIF immunoreactivity in non-medicated PD may be PD-specific since no detectable changes were found in the CSF of cases affected by progressive supranuclear palsy (PSP), a neurodegenerative disorder which at early stages shares common clinical features with PD (FIG. 11). Excitingly, using “receiver operating characteristic” statistical analysis, we were able to distinguish PD cases from the control cases with 81% sensitivity and 100% specificity. We confirmed the specificity and sensitivity of PIF to detect PD (medicated and non-medicated) from neurological controls across multiple cohorts.

FIG. 11 shows the rabbit anti-human PIF95-109 antibody was used in a single antibody ELISA assay to quantify the content of PIF immunoreactivity in the CSF of non-medicated PD (n=27) from the DATATOP study collection and neurological normal control (n=11) cases, provided through a collaboration with Dr. LeWitt (Collaborator) or PSP (n=14) cases, provided through an ongoing collaboration with Dr. Irene Litvan, (Henry M. Jackson Foundation, Bethesda). For this single antibody ELISA assay, microtiter wells were coated with the antigen (0.5 μl CSF diluted 200-fold) dissolved in 100 μl of 50 mM carbonate buffer, pH 9.6, and incubated at 4° C., overnight. All subsequent incubations were made in a volume of 50 μl/well. The plates were blocked with dilution buffer (10 mM Tris-HCl buffer, pH 7.4, containing 0.05% Tween 20, 0.5 M NaCl and 5% skimmed milk) followed by incubation with the affinity-purified rabbit anti-PIF95-109 antibody (purified by affinity chromatography using the immunizing synthetic peptide) for 1 h at room temperature. The plates were incubated with biotin-conjugated anti-rabbit 1 gG in dilution buffer for 1 h and then incubated with horseradish peroxidase-conjugated streptavidin (Amersham Biosciences) for 15 min. Color was developed with 3,3′,5,5′-tetramethyl-benzidine (0.4 mg/ml) in 0.05 M citric acid phosphate buffer, pH 5.0, containing H₂O₂ (0.006

Exogenous Application of Human Recombinant PIF (hrPIF) Potentiates MPP⁺-Mediated DAergic Neurodegeneration in vitro

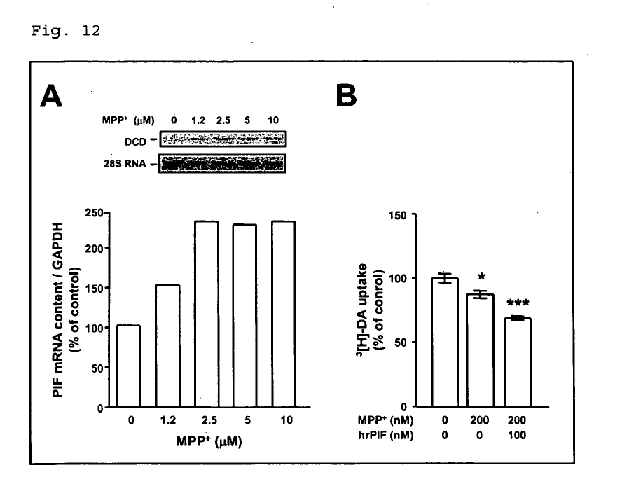

The exact molecular mechanisms leading to the pathophysiology of PD are not well understood. MPP⁺, a mitochondrial complex I inhibitor, produces PD-type symptoms in humans and laboratory animals and has been used to investigate PD pathogenesis^{12,13}. MPP⁺, the ultimate metabolite of MPTP, is taken up into DAergic neurons where it accumulates in mitochondria and works to inhibit complex I activity. Although the mechanism of MPP⁺-induced neurotoxicity is not fully understood, there is increasing evidence supporting the involvement of reactive oxygen species (ROS)¹⁴. Based on this consideration, we continued to explore the regulation of PIF expression in response to MPP⁺-induced neurotoxic injury. Excitingly, we found that MPP⁺dose dependently induced PIF mRNA expression in enriched rat DAergic neuron cultures (FIG. 12A) . Based on this evidence, we continued to test the hypothesis that PIF promotes MPP⁺-mediated neurotoxicity in DAergic cell culture. For this study, hrPIF was generated using M15 bacterial cells harboring His-tagged PIF expression construct (gift from Dr. Polyak, Co-investigator) . HrPIF was purified as previously reported¹⁵ and the purity of hrPIF was confirmed by SDS-PAGE and western blot analyisis using rabbit anti-PIF antibody¹⁵. Interestingly, we found that exogenous application of hrPIF to rat DAergic neuron cultures promoted a ˜1.5 fold potentiation of MPP⁺-mediated neurotoxicity in an autocrine/paracrine manner, as assessed by dopamine uptake assay (FIG. 12B). This preliminary result is of great interest and provides initial evidence that a novel biomarker whose content is elevated in clinical PD, such as PIF, might be also involved in PD pathophysiology as discussed below.

For these studies enriched mesecephalic DAergic cultures were obtained from E16 Sprague Dawley embryos and cultured for 10 days in poly 1-ornithine coated culture dish at a density of 0.5×10⁶ cells per well and maintained in Neurobasal medium with B-27 supplement and 0.5 mM glutamine, as previously described¹⁶. In A, following MPP⁺ treatment for 12 hr, total RNA was extracted from the neuronal cultures using Ultraspec RNA (Biotecx) and PIF mRNA expression assessed by northern blot hybridization assay using ³²p labeled hPIF cDNA¹⁷ and quantified by phospho-imaging (Molecular Dynamics). In B, cultures were treated with MPP⁺ in the presence/absence of PIF 12 hours, at the concentrations indicated. Uptake studies with [³H]-dopamine were performed as described by Storch et al.¹⁸. Non-specific transport was determined in parallel assays conducted in the presence of 1 uM GBR12909 and incorporated [³H]-dopamine was extracted with NaOH (0.5M) and quantified by liquid scintillation spectrometry

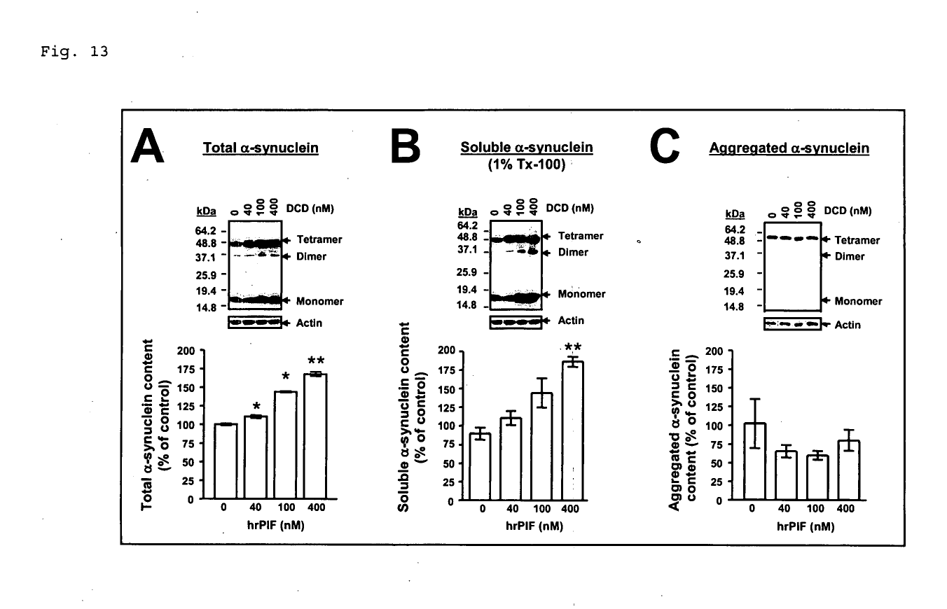

Because of the recent evidence showing increased α-synuclein expression increases vulnerability to MPP/MPTP¹⁹, we decided to explore the possibility that PIF might have promoted DAergic neurodgeneration through mechanisms that involve α-synuclein expression. Encouragingly, we found that hrPIF-mediated potentiation of MPP⁺-induced neurotoxicty coincided with increased expression of total α-synuclein (FIG. 13A) leading to an accumulation of soluble oligomeric α-synuclein protofibrils (FIG. 13B), but not aggregated α-synuclein (FIG. 13C). Our results are especially relevant in consideration of another finding that showed overexpression of wild-type α-synuclein can mediate neurotoxicty²⁰. Moreover, a recent study in humans showed that a triplication of the chromosome containing α-synuclein results in familial PD²¹. This evidence is also consistent with work in Drosophila where overexpression of α-synuclein leads to DAergic toxicity²².

In these studies (see FIG. 13) 10 days old enriched mesecephalic DAergic cultures were obtained as discussed in FIG. 12. In A, following treatment with increasing concentrations of hrPIF, cultures lysed in each well using SDS-PAGE (5 mM Tris-HCl, 2% SDS, 0.1 M DTT, 0.001% Bromphenol blue, 10% glycerol, pH6.8) sample buffer. In B and C, following hrPIF treatment, cultures were rinsed twice with ice-cold PBS and gently lysed directly in the well with buffer T (20 mM Tris-HCl, pH 7.4, 25 mM KCl, 5 mM MgCl₂, 0.25 mM Sucrose, 1% Triton X-100, proteases inhibitor cocktails). After 5 min incubation at RT in buffer, the Triton X-100 soluble fraction was gently collected from the-dish-. -The remaining Triton-insoluble material was gently rinsed once with ice cold PBS, and collected in buffer N (0.1 M Na₂CO₃, pH 11.5 with protease inhibitor cocktails). In C, the Triton-insoluble synuclein fraction was collected in the pellet by centrifugation at 16,000 g for 10 min and dissolved in 1×SDS buffer. Immunoblots used monoclonal anti α-synuclein antibody (1:1000, BD Bioscience) and immunoreactivities were visualized densitometrically.

In view of the evidence that elevation of soluble oligomeric □-synuclein protofibrils precedes the formation of insoluble □-synuclein inclusions found in PD brain, our studies for the first time suggest that PIF may be a novel risk factor in PD α-synucleopathy.

Elevated α-Synuclein Expression in the Brain of hPIF Transgenic Mice

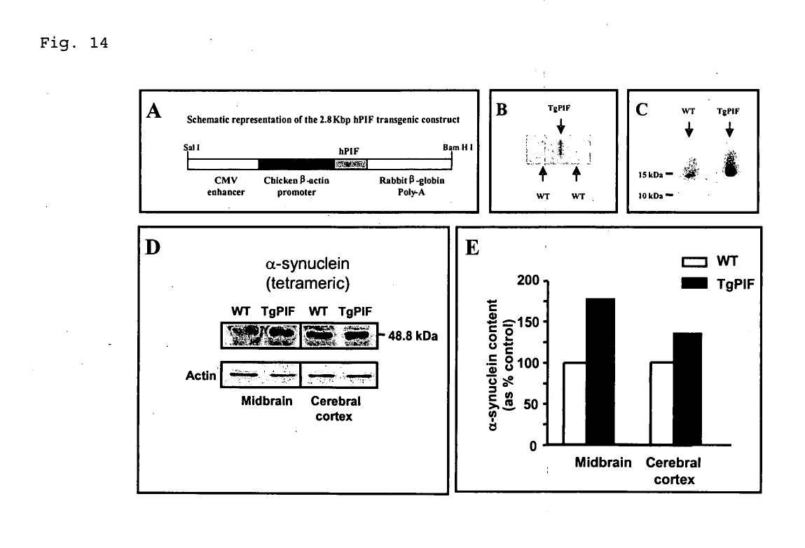

Based on the evidence that hrPIF can exacerbate MPP⁺-induced neurotoxicity in enriched DAergic cultures in correlation with an elevation of soluble oligomeric α-synuclein protofibrils, we initiated a series of studies to further explore the role of PIF in MPTP-stimulated potentiation of α-synuclein protofibril levels in hPIF transgenic (TgPIF) mice. Preliminary studies in the lab confirmed that ubiquitous expression of hPIF (driven by a beta-actin promoter) (FIG. 14A) results in a ˜3 fold elevation (quantification not shown) of PIF steady-state levels in the serum as assessed by western blot assay (FIG. 14C). The increase in serum levels of PIF correlated with a ˜2 fold elevation of α-synuclein expression in the midbrain and lesser extend in the cerebral cortex (and other brain regions, not shown) relative to wild-type controls (FIGS. 14D,E). These in vivo results support our in vitro findings showing that hrPIF promotes the generation of soluble α-synuclein protofibrils in enriched DAergic neuron cultures. Thus, using our TghPIF mouse model we will test the hypothesis that ubiquitous overexpression of PIF may promote DAergic neurodegeneration in response to MPTP, possibly through mechanisms that involves the generation of α-synuclein expression in the brain in a autocrine-pararcrine manner as found in vitro.

For the generation of hPIF trangenics a 458-bp CDNA fragment containing the entire coding region for human (h)PIF CDNA flanked by a 5′-EcoR I and a 3′-BamH I restriction site was generated by PCR amplification from human skin cDNA using the primer set: 5′-attagaattcgaccctagatcccaagatc-3′ and 5′-attaggatccaggttttaggctgaagacg-3′ (see FIG. 14). After EcoR I and BamH I digestion, the hPIF cDNA was cloned into the EcoR I and Bgl II sites of the pCAGGS vector and the 2.8 Kbp hPIF transgenic construct was isolated following digestion with Sal I and BamH I. For generation of hPIF-transgenics, the hPIF transgenic construct was gel-purified and microinjected into one-cell mouse eggs as previously described²³, using C57Bl/6×C3H (B6C3) Fl hybrid mice (Taconic Farms) as the source of fertilized eggs at the pronuclear stage. Transgenic mice produced were identified by dot blot hybridization of tail skin DNA samples with a ³²P-labeled random-primed cDNA probe generated from the entire 2.8 kBp hPIF transgenic construct as previously described²³. Panel 14B, identification of a transgenic hPIF (TgPIF20) founder mouse by tail DNA dot-blot hybridization. TgPIF20 founder was mated with wild-type mice and Fl offspring were sorted by genotyping. Panel 14C, western blot analysis using rabbit-anti human PIF53-64 antibody from our lab (1:5000) confirmed increased steady state levels of hPIF protein content in serum of 5 month old TgPIF transgenic compared to age matched WT littermates. The primary elevated form of hPIF found in the serum of our TgPIF mice ranged ˜16 kDa which is consistent with previous evidence of multiple (secreted) glycosylated PIF forms ranging 14-20 kDa in human cells. Panel 14D, total α-synuclein (tetramer) immunoreactivity in the midbrain and cerebral cortex of TgPIF and WT littermates were quantified densitometrically in Panel 14E. For the western gel blot analysis of total α-synuclein shown in D, brain tissue was extracted in standard RIPA buffer containing 0.1% SDS.

Characterization of PIF Receptor in Human Brain

PIF Receptor Binding is Preferentially Localized in Midbrain SN Neurons

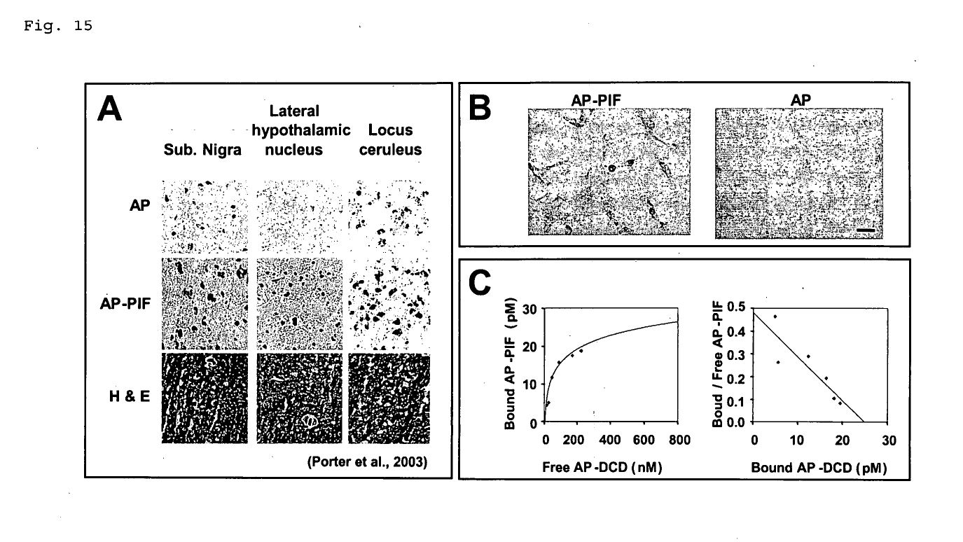

In view of the evidence that PIF is a functionally secreted glycoprotein^{3,5} and the fact that its function could by mediated through binding to a cell-surface receptor in peripheral organs, we initiated a series of studies to explore the distribution of the PIF receptor in the brain. For these studies, in collaboration with Dr. Polyak (Co-Investigator), we generated an N-terminal alkaline phosphates (AP) C-terminal PIF precursor protein fusion protein as a ligand. This ligand was then used in receptor binding assays in situ¹⁵. As shown in FIG. 15A (and illustrated in Porter et al., 2003¹⁵), we found that in human brain high-intensity AP-PIF receptor binding was primarily localized to midbrain substantia nigra, hypothalamus, and locus ceruleus relative to AP background signal on adjacent tissue control sections (FIG. 15A); this evidence indicating a functional receptor binding activity in brain regions at high risk for PD degeneration is consistent with the hypothesis that PIF may promote PD type neuropathology, possibly through α-synuclein-mediated mechanisms. Based on the evidence that PIF binding activity is localized to brain regions at high risk for neurodegeneration in PD we further characterized the PIF receptor. As an initial characterization of PIF cell surface receptor, we surveyed cultured cell line for expression of PIF receptor. Excitingly, we identified a single high-affinity PIF cell surface receptor in the human SH-SY5Y neuronal cell line (FIG. 15B), with an apparent dissociation constant of 5.1×10⁻⁸ M, and maximum binding concentration of 30,000 PIF receptors per cell (FIG. 15C).

References

1. Song, D. D., Shults, C. W., Sisk, A., Rockenstein, E. & Masliah, E. Enhanced substantia nigra mitochondrial pathology in human alpha-synuclein transgenic mice after treatment with MPTP. Exp. Neurol. 186, 158-172 (2004).

2. Sharon, R. et al. The formation of highly soluble oligomers of alpha-synuclein is regulated by fatty acids and enhanced in Parkinson's disease. Neuron 37, 583-595 (2003).

3. Todorov, P. T. et al. Induction of muscle protein degradation and weight loss by a tumor product. Cancer Res. 56, 1256-1261 (1996).

4. Todorov, P. T., Field, W. N. & Tisdale, M. J. Role of a proteolysis-inducing factor (PIF) in cachexia induced by a human melanoma (G361). Br. J. Cancer 80, 1734-1737 (1999).

5. Todorov, P. et al. Characterization of a cancer cachectic factor. Nature 379, 739-742 (1996).

6. Cunningham, T. J., Jing, H., Wang, Y. & Hodge, L. Calreticulin binding and other biological activities of survival peptide Y-P30 including effects of systemic treatment of rats. Exp. Neurol. 163, 457-468 (2000).

7. Cunningham, T. J. et al. Identification of a survival-promoting peptide in medium conditioned by oxidatively stressed cell lines of nervous system origin. J. Neurosci. 18, 7047-7060 (1998).

8. Conway, K. A., Harper, J. D. & Lansbury, P. T., Jr. Fibrils formed in vitro from alpha-synuclein and two mutant forms linked to Parkinson's disease are typical amyloid. Biochemistry 39, 2552-2563 (2000).

9. Stoppini, M., Andreola, A., Foresti, G. & Bellotti, V. Neurodegenerative diseases caused by protein aggregation: a phenomenon at the borderline between molecular evolution and ageing. Pharmacol. Res. 50, 419-431 (2004).

10. Wersinger, C., Banta, M. & Sidhu, A. Comparative analyses of alpha-synuclein expression levels in rat brain tissues and transfected cells. Neurosci. Lett. 358, 95-98 (2004).

11. Wolozin, B. & Golts, N. Iron and Parkinson's disease. Neuroscientist. 8, 22-32 (2002).

12. Langston, J. W. The etiology of Parkinson's disease with emphasis on the MPTP story. Neurology 47, S153-S160 (1996).

13. Sawada, H. et al. Proteasome mediates dopaminergic neuronal degeneration, and its inhibition causes alpha-synuclein inclusions. J. Biol. Chem. 279, 10710-10719 (2004).

14. Schmidt, N. & Ferger, B. Neurochemical findings in the MPTP model of Parkinson's disease. J. Neural Transm. 108, 1263-1282 (2001).

15. Porter, D. et al. A neural survival factor is a candidate oncogene in breast cancer. Proc. Natl. Acad. Sci. U. S. A 100, 10931-10936 (2003).

16. Lee, H. J., Khoshaghideh, F., Patel, S. & Lee, S. J. Clearance of alpha-synuclein oligomeric intermediates via the lysosomal degradation pathway. J. Neurosci. 24, 1888-1896 (2004).

17. Schittek, B. et al. Dermcidin: a novel human antibiotic peptide secreted by sweat glands. Nat. Immunol. 2, 1133-1137 (2001).

18. Storch, A. et al. Dopamine transporter-mediated cytotoxicity of beta-carbolinium derivatives related to Parkinson's disease: relationship to transporter-dependent uptake. J. Neurochem. 89, 685-694 (2004).

19. Robertson, D. C. et al. Developmental loss and resistance to MPTP toxicity of dopaminergic neurones insubstantia nigra pars compacta of gamma-synuclein, alpha-synuclein and double alpha/gamma-synuclein null mutant mice. J. Neurochem. 89, 1126-1136 (2004).

20. Petrucelli, L. et al. Parkin protects against the toxicity associated with mutant alpha-synuclein: proteasome dysfunction selectively affects catecholaminergic neurons. Neuron 36, 1007-1019 (2002).

21. Singleton, A. B. et al. alpha-Synuclein locus triplication causes Parkinson's disease. Science 302, 841 (2003).

22. Auluck, P. K., Chan, H. Y., Trojanowski, J. Q., Lee, V. M. & Bonini, N. M. Chaperone suppression of alpha-synuclein toxicity in a Drosophila model for Parkinson's disease. Science 295, 865-868 (2002).

23. Kelley, K. A. et al. Potentiation of excitotoxicity in transgenic mice overexpressing neuronal cyclooxygenase-2. Am. J. Pathol. 155, 995-1004 (1999).

24. Flanagan, J. G. & Leder, P. The kit ligand: a cell surface molecule altered in steel mutant fibroblasts. Cell 63, 185-194 (1990).

25. Kolodkin, A. L. et al. Neuropilin is a semaphorin III receptor. Cell 90, 753-762 (1997).

1. A method of assessing the likelihood that a patient is suffering from Parkinson's disease which comprises:
(a) obtaining a fluid sample from the subject;
(b) contacting the sample with an agent which forms a complex with a protein comprising consecutive amino acids having the sequence set forth in SEQ ID NO:1, 2, or 4, or a fragment thereof, under conditions permitting any such protein or fragment thereof present in the sample to complex with the agent; and
(c) detecting the presence of any protein-agent complex formed in step (b),
wherein the detection of protein-agent complex in step (c) indicates that the patient is likely suffering from Parkinson's disease.

2. The method of claim 1, wherein the sample is cerebrospinal fluid.

3. The method of claim 1, wherein the sample is a derivative of cerebrospinal fluid.

4. (canceled)

5. (canceled)

6. The method of claim 1, wherein the agent is an antibody.

7. The method of claim 6, wherein the antibody is a monoclonal antibody.

8. (canceled)

9. The method of claim 6, wherein the antibody is a labeled antibody and wherein the detecting of the presence of protein-agent complex is effected by detecting the label on the antibody.

10. (canceled)

11. The method of claim 1, wherein the patient is suffering from a neurodegenerative disease.

12. A method of assessing the likelihood that a patient is susceptible to suffering from Parkinson's disease which comprises:
(a) obtaining a fluid sample from the subject;
(b) contacting the sample with an agent which forms a complex with a protein comprising consecutive amino acids having the sequence set forth in SEQ ID NO:1, 2, or 4, or a fragment thereof, under conditions permitting any such protein or fragment thereof present in the sample to complex with the agent; and
(c) detecting the presence of any protein-agent complex formed in step (b),
wherein the detection of protein-agent complex in step (c) indicates that the patient is likely susceptible to suffering from Parkinson's disease.

13. The method of claim 12, wherein the sample is cerebrospinal fluid.

14. (canceled)

15. (canceled)

16. (canceled)

17. (canceled)

18. (canceled)

19. (canceled)

20. (canceled)

21. A method of assessing the likelihood that a patient is suffering from Parkinson's disease which comprises:
(a) obtaining a fluid sample from the subject;
(b) contacting the sample with an agent which forms a complex with a human DCD-1 protein comprising consecutive amino acids having the sequence set forth in SEQ ID NO: 1, 2, or 4, or homolog thereof, under conditions permitting any such protein or homolog thereof present in the sample to complex with the agent; and
(c) detecting the presence of any protein-agent complex formed in step (b),
wherein the detection of protein-agent complex in step (c) indicates that the patient is likely susceptible to suffering from Parkinson's disease.

22. The method of claim 21, wherein the sample is cerebrospinal fluid.

23. (canceled)

24. (canceled)

25. (canceled)

26. (canceled)

27. (canceled)

28. (canceled)

29. (canceled)

30. (canceled)

31. (canceled)

32. The method of claim 1, further comprising determining the amount of complex formed in step (b) and comparing such amount with a standard, wherein a greater amount of complex formed in step (b) than in the standard indicates that the subject is likely suffering from Parkinson's disease.

33. The method of claim 12, further comprising determining the amount of complex formed in step (b) and comparing such amount with a standard, wherein a greater amount of complex formed in step (b) than in the standard indicates that the subject is likely suffering from Parkinson's disease.

34. The method of claim 21, further comprising determining the amount of complex formed in step (b) and comparing such amount with a standard, wherein a greater amount of complex formed in step (b) than in the standard indicates that the subject is likely suffering from Parkinson's disease.

35. A method of assessing the likelihood that a patient is suffering from Parkinson's disease which comprises:
(a) providing a solid support to which an agent which forms a complex with a human DCD-1 protein comprising consecutive amino acids having the sequence set forth in SEQ ID NO: 1, 2, or 4, under conditions permitting any human DCD-1 protein present in the sample to complex with the agent is bound;
(b) contacting the solid support from (a) with a fluid sample from the subject;
(c) removing any of the human DCD-1 protein which is not bound to the solid support; and
(d) detecting the presence of protein bound to the solid support,
wherein the detection of the human DCD-1 protein bound to the solid support in step (d) indicates that the patient is likely suffering from Parkinson's disease.

36. The method of claim 35, wherein the sample is cerebrospinal fluid.

37. (canceled)

38. (canceled)

39. (canceled)

40. The method of claim 35, wherein the agent is an antibody.

41. (canceled)

42. (canceled)

43. The method of claim 35, wherein the agent is a labeled antibody and wherein the detecting of the presence of protein/labeled antibody complex is effected by detecting the label.

44. The method of claim 43, wherein the label is a radioisotope, a chromophore, a biomolecule, a fluorophore, a radiolabeled molecule, a dye, an affinity label, an antibody, biotin, streptavidin, a metabolite, a mass tag, or a dextran.

45. The method of claim 35, wherein the patient is suffering from a neurodegenerative disease.

46. (canceled)

47. (canceled)

48. (canceled)

49. (canceled)


SEQUENCE LISTING (4 sequences)

SEQ ID NO: 1 - protein, 47 aa, homo sapiens
SSLLEKGLDGAKKAVGGLGKLGKDAVEDLESVGKGAVHDVKDVLDSV

SEQ ID NO: 2 - protein, 110 aa, homo sapiens
MRFMTLLFLTALAGALVCAYDPEAASAPGSGNPCHEASAAQKENAGEDPGLARQAPKPRKQRSSLLEKGLDGAKKAVGGLGKLGKDAVEDLESVGKGAVHDVKDVLDSVL

SEQ ID NO: 3 - DNA, 458 bp, homo sapiens
gaccctagatcccaagatctccaaggatttggtggcatacccactccagcacacagaagcatgaggttcatgactctcctcttcctgacagctctggcaggagccctggtctgtgcctatgatccagaggccgcctctgccccaggatcggggaacccttgccatgaagcatcagcagctcaaaaggaaaatgcaggtgaagacccagggttagccagacaggcaccaaagccaaggaagcagagatccagccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagtactatagctgtaaggagaagctgagaaatgatacccaggagcagcaggctttacgtcttcagcctaaaacct

SEQ ID NO: 4 - protein, 11 aa, homo sapiens
DAVEDLESVGK